(19) United States
(12) Patent Application Publication
(10) Pub. No.: US 2014/0018634 A1
(43) Pub. Date: Jan. 16, 2014

(54) Method of determining skin type, choosing skin care products and procedures and promoting skin care products

(71) Applicant: Leslie S. Baumann, Miami Beach, FL (US)

(72) Inventor: Leslie S. Baumann, Miami Beach, FL (US)

(21) Appl. No.: 14/025,546

(22) Filed: Sep. 12, 2013

Related U.S. Application Data
(63) Continuation of application No. 11/283,511, filed on Nov. 21, 2005, now abandoned.

(51) Int. Cl. A61B 5/00

(52) U.S. Cl. CPC A61B 5/441
USPC 600/300; 434/322

(57) ABSTRACT

The invention comprises a system and method for assigning a skin type and/or hair type to an individual, where a preferred example is one of sixteen hair types and one of sixteen skin types. The hair or skin type score uses at least four factors related to the condition of the skin or hair, or a section of it. The invention also comprises using the hair type and skin type scores assigned to an individual to recommend or suggest certain skin and/or hair care products, as well as skin and/or hair care products that can be avoided. A preferred embodiment employs the skin or hair type in an online marketing, web-based sales network, or other computer-implemented or electronic interface, so that customers can use skin or hair type scores to receive information about products, ingredients, or to buy recommended products in a variety of ways.

### RELATED APPLICATIONS

This application claims the benefit of prior U.S. provisional application 60/629,320, filed Nov. 19, 2004, the entire contents of which are hereby incorporated by reference.

### FIELD OF THE INVENTION

The invention relates to skin and/or hair care methods, products, treatments, and educational, promotional, and diagnostic methods concerning them and the analysis of skin and/or hair types. By employing a new classification scheme that better identifies the basis for skin and hair care treatments and the physiological and cellular differences between human skin and/or hair types, the invention provides advantageous results and leads to more informed cosmetic and skin and hair care decisions by professionals, consumers, or patients. In one aspect of the invention, patients, consumers, manufacturers, physicians, aestheticians, hair stylists, nurses and others can stratify an individual's skin or hair into 16 unique skin and/or hair types based on values assigned to at least four parameters. This results in 16 separate skin types and 16 separate hair types, referred to as Baumann Skin Type or Baumann Hair Type. The 16 different skin or hair types can be used to improve marketing and product and treatment recommendations.

### RELATED ART AND BACKGROUND TO THE INVENTION

In order to promote skin and hair care products, many companies ask potential customers questions regarding their perception of their skin and hair. Based on the answers to these questions, cosmetic companies are able to better suggest skin and hair care products. Examples of skin care promotions and these questions can be found on the Internet webpages of Neutrogena (neutrogena.com), L'Oreal (or L'Oreal website lorealparisusa.com), and Lancome (or Lancome website lancome.com). The answers to these questions, however, merely tell patients that their skin type is either dry, combination, sensitive, or oily. Similarly, hair care promotions that discuss hair types focus on fine, or curly, or color treated hair (see L'Oreal website at lorealparisusa.com). Some promotional efforts and websites, such as Pantene (website pantene.com), focus on a hair care objectives, for example increasing volume. In addition, many patent documents discuss questionnaires and testing methods in an attempt to categorize skin or hair, such as US patent publication 2004/0122299.

Each of these skin and hair care promotional efforts, as well as related websites and/or product literature that discusses skin and hair types, are limited to one or perhaps two parameters. As a result, the methods cannot effectively suggest or recommend products that address all of the skin and hair conditions that can exist.

In addition, there are two skin type classification systems that are currently used in the dermatology field. The first is based on the skin's reactivity to the sun and was developed by Fitzpatrick in 1963 (Fitzpatrick T B, Breathnach A S; Das Epidermal Melanin-Einheit System, Dermatol Wochenschr 147:481-489 (1963)). This grading scale is used universally but only takes into account the skin's pigmentation and reaction to sun exposure. More recently, a scale was developed to rank the degree of photodamage or skin aging caused by the sun. This “Glogau Photoaging scale” divides skin into 4 types according to the amount of wrinkles that are present (Glogau, R G, Aesthetic and Anatomic Analysis of the Aging Skin, Semin Cutan Med Surg, 15(3):134-8 (1996)). There are no widely accepted skin typing systems that take into account wrinkles, pigmentation, dryness and sensitivity. In addition, there are no widely used systems to type hair. Thus, the dermatology field is in need of improved methods for categorizing skin and hair types and recommending products for certain skin and hair types. There is, accordingly, a need in the art for more effective and more particular stratification of skin types and hair types as well as improved methods for determining the appropriateness of products for treating particular skin and hair.

### SUMMARY AND DESCRIPTION OF THE INVENTION

In one aspect, the invention comprises a skin type scoring system and skin typing nomenclature that can involve a combination of: (i) a questionnaire to score skin for at least 4 factors or parameters; (ii) a list of 16 skin types based on the score in each of the four factors or parameters; and (iii) any other method that determines one of the four parameters, or a patient's self-perception of that parameter, to assign one of the 16 skin types. These 16 skin types can be used as an output for recommending personal care products and/or treatments that can be used to treat or care for skin having wrinkles, pigmentation, dryness and sensitivity, for example. In another aspect, the invention comprises a hair type scoring system and hair typing nomenclature that can involve a combination of: (i) a questionnaire to score hair for at least 4 factors or parameters; (ii) a list of 16 hair types based on the score in each of the four factors or parameters; and (iii) any other method that determines one of the four parameters, or a patient's self-perception of that parameter, to assign one of the 16 hair types. These 16 hair types can be used as an output for recommending personal care products and/or treatments that can be used to treat or care for hair having chemical damage, a fine diameter, dryness and curls, for example. Thus, the invention allows patients, consumers, manufacturers, physicians, aestheticians, hair stylists, nurses and others to stratify skin or hair types into 16 unique skin and/or hair types based on values assigned to at least four parameters. In certain embodiments, this results in sixteen separate skin types and/or sixteen separate hair types, referred to as Baumann Skin Type or Baumann Hair Type.

While sixteen skin types are noted in particular and specifically here, the four factors or parameters may have a larger “score” range and, thus, instead of a being oily or dry (where the degree or score is one of two possible), the score or degree will depend upon the score on the questionnaire. For example, on the oily or dry portion of the questionnaire, the results may be oily1 or oily2 or dry1 or dry2 (totaling four different scores), for example. Furthermore, the number of parameters or factors can be increased over four. The number of possible scores or the range of scores for each of the four (or more) parameters or factors can depend on the input or answers to questions related to each of the parameters and the value of increasing the range of scores has in predicting an optimum skin care product or treatment or ingredient. If, for example, one of the available methods for analyzing a parameter is used and can produce results or input for that parameter that can increase the ability to differentiate between a recommended product and a neutral product or an unhelpful product or a contra-indicted product (“avoid” product), the score or range of scores can be selected and used in the invention.

The method of the invention can incorporate existing or any available method or technique for evaluating the four parameters including the enclosed questionnaire. Thus, the Glogau photoaging scale can be used for determining an aspect of the parameter involving wrinkles and the degree or score for that parameter can be associated with one of the four types identified by Glogau, or one of the six types of the Fitzpatrick-Pathak chart for sensitivity to sunlight. Many similar scales and scoring methods can be incorporated into the invention and its use for the wrinkled/tight factor and/or the sensitive/resistant parameter. In addition, each of the following references can be used to select a scoring system or method of inputting a score for a particular parameter in the methods of the invention, and each document is specifically incorporated by reference in its entirety and can be used and relied on to make specific embodiments of the invention: Baumann, Cosmetic Dermatology: Principles and Practice, McGraw-Hill (2002); Glogau, Semin Cutan Med Surg 15: 134-8 (1996); Glogau, J., Geriatr Dermatol 12:31 (1994); Armstrong, et al., Arch Dermatol 128:390-3 (1992); Williams, Arch Dermatol 128:1406-7 (1992); Griffiths, et al., Arch Dermatol 128:347-51 (1992); Holck, Curr Opin Ophthalmol 14:246-52 (2003); Perednia, et al., Arch Dermatol 128:357-64 (1992). Other methods and techniques can also be selected and used.

In a particular embodiment, the invention comprises a method of identifying a skin care, hair care, or personal care product for a user or recommending a skin care, hair care or personal care product to a user, patient, or professional, wherein the skin or hair, or a portion or section of the skin or hair, of a patient is scored for at least four factors or parameters. For each factor, the score is compared to information on a group of skin care, hair care, or personal care products, which information can be stored in electronic form, in a database, in a printed publication, in a chart, or in other medium. The information can be continually updated to enter new products or new characteristics of products. Examples of the information or characteristics for the products and/or treatments are specific indications that the product is designed to be used for dry skin, or oily skin, or that it contains allergens or compounds that users having sensitive skin should avoid, or that the product should not be used when the user will be exposed to the sun, or that the product tends to cause discoloration in certain skin, etc. The method can further comprise selecting at least one skin care product for the user and/or optionally recommending at least one product for purchase and/or recommending the use of that at least one product or a specific regimen for use and/or recommending that at least one product NOT be used or purchased. In the case of skin typing, the method can be further refined by using a particular set of parameters or factors, such as: 1) the degree of oily or dry skin present (O or D); 2) the degree of sensitive or resistant skin present (S or R); 3) the degree of pigmented or non-pigmented skin present (P or N); and 4) the degree of wrinkled or tight skin present (W or T). Thus, a particular user can be defined as having oily, sensitive, pigmented, and tight skin (OSPT) or any other of the sixteen possible combinations using these particular factors. The one of sixteen separate skin types that can be assigned to a user or patient can be referred to as the Baumann Skin Type. In the case of hair typing, the method can be further refined by using a particular set of parameters or factors, such as: 1) the degree of oily or dry hair present (O or D); 2) the degree of injured or uninjured hair present (I or U); 3) the degree of small diameter (fine) or large diameter (thick) hair present (F or T); and 4) the degree of curly or straight hair present (C or S). Thus, a particular user can be defined as having dry, injured, fine, curly hair (DIFC) or any other of the sixteen possible combinations using these particular factors. The one of sixteen separate hair types that can be assigned to a user or patient can be referred to as the Baumann Hair Type. Thus, in one aspect of this embodiment of the invention, the method encompasses assigning one of sixteen Baumann Skin Types and/or Hair Types to a user or to a user's skin or hair or portion thereof. The information on cosmetic or skin or hair or personal care products, or the particular ingredients in those products, can then be used to recommend one or more products or treatments, or recommend against one or more products or treatments, for a particular skin type or condition characterized by a particular skin type. Similarly, a classification of sixteen hair types using these four parameters: dry or oily scalp; injured treated or non-injured treated; fine hair or thick hair; curly hair or straight hair.

Additional factors or parameters can be selected and used, or others substituted for those four listed above, but the four factors are preferred. If the four factors noted here are selected and used, the method can be further refined by assigning a range or score for each factor, so that, for example, factor 1) is a range from the integers 1-4 and a score of 1 in the oily to dry factor represents dry skin and a score of 4 represents oily whereas the integers in between represent lesser degrees of dryness or oiliness. Similarly, the score for factor 2) can be a range from the integers 1-4, so that, for example, a score of 1 in the sensitive to resistant factor represents sensitive and a score of 4 represents resistant, and the score for factor 3) is a range from the numbers 1-4, so that, for example, a score of 1 in the pigmented or non-pigmented factor represents non-pigmented skin and a score of 4 represents pigmented skin, and the score for factor 4) is a range from the numbers 1-4, so that, for example, a score of 1 in the wrinkled or tight factor represents tight skin and a score of 4 represents wrinkled skin. Of course, other ranges can be selected, but in this example a 4×4×4 grid of the four factors and the score for each factor can be generated. Each place on the grid (for example O1:S1:P1:T1) can then be matched with a recommended product, treatment, ingredient, or combination of ingredients in the same or similar manner that the OSPT example from the sixteen skin type example above.

In another aspect of the invention, the method can operate on or as a computer-based or network system, such that, for example, the score for each factor and/or the input of the scores and patient or user information can be performed via a terminal or computer or through an access to an online network. Thus, a patient or user input in each of four parameters or factors, for example, gives rise to one or more recommendations in skin or personal care products or ingredients or combinations of ingredients or even treatments or uses of a product. In addition, the method can be used specifically to indicate, recommend, or suggest a particular skin care product, a cosmetic product, a cosmetic product for women, a cosmetic product for men, a cosmetic product for children or infants, a particular treatment for a skin disease or disorder, a hair care product, or a combination of any of these. The method can also be used in diagnosing a skin disease or disorder.

In another aspect, the collection or database of product and ingredient information can be stored electronically on the computer-based system or network and accessed to provide output in recommending a personal care product or treatment regimen. The collection of information or data on products or treatments can focus on the product line of one particular company or producer, a group of companies or producers, a particular ingredient or combination of ingredients, or encompass many different companies or producers or ingredients or combinations. Furthermore, the information, or the output from the collected information or database, can be limited to those products available at a particular store or through a particular merchant or at a particular location or even to a particular country or region within a country. It can also be limited to those products or treatments that can be sold over-the-counter or used without professional guidance or direction or without a doctor's supervision.

As noted, in any embodiment of the invention the information about the products or treatments can be compiled in a list, chart, document or electronic database or accessible database. Each of the products or treatments and the method of using each of them can be categorized by one or more values according to the possible skin or hair types being used. Thus, for example, the record or datapoint for each product or treatment associated with a particular skin or hair type can also store information (or be linked to information or comments) for the user or queries to the user to further define the product, the user, or the skin type. Thus, in a general aspect, the invention allows an interactive computer-based or network system for determining a user's skin or hair type or the particular product or products to be recommended for that user.

The data or information for each product can be sub-categorized by the ingredients in that product and/or by the function of the product or ingredient(s). Methods and techniques for determining the ingredients are known in the art and one can refer to texts (for example, Cosmetic Analysis: Selective Methods and Techniques, P. Bore, ed., C.H.I.P.S., 2005) in order to select such methods or techniques. Thus, certain products that contain certain ingredients or combinations of ingredients may be recommended or not recommended for a particular skin or hair type.

In another embodiment, a questionnaire or series of questions can form the user or patient input in the method, which can be referred to as the Baumann Skin Type Questionnaire or the Baumann Hair Type Questionnaire. If the degree or score for a parameter or factor is between one of two possibilities, the questionnaire can essentially be a series of questions. Optionally, and especially if the degree or score for a parameter or factor is between more than one of two possibilities, the questions or queries can be a flow chart or set of questions or even an interactive set of questions, where a response to a question is followed by one or more questions to refine the answer or input for a particular factor or parameter. In addition, the input or answers to a question, set or questions, or a query as from an interactive network is the value from one of the analytical methods, available scaling systems, or techniques noted above, for example the Glogau photoaging scale.

In addition, the invention can incorporate a patient or user (patient/customer) information collection step or process, where a user can save identifying information or a username to be retrieved or used later. The patient/customer information can include basic demographic queries and/or responses as well as prior user sessions and/or recommendations. Systems and networks that utilize this patient/customer information or demographic information are well known in the art, as well as the methods for sequestering this personal information to protect the privacy of the user or patient or customer.

### BRIEF DESCRIPTION OF THE FIGURES

FIG. 1 depicts an exemplary system or method for receiving skin or hair type information input from a terminal, network, local area network, or via the internet, for example, identifying one or more products to be recommended and/or one or more products to be avoided based upon the skin or hair type, and creating an output based upon the products and the information about the products stored in the identifier. In optional embodiments, the output can be sent to a printer or screen for display by a potential customer or user. In addition, optional embodiments can associated a user ID for each individual customer or user, associate a user ID with that individual's skin or hair type, and store “Preferences” information on products or treatments that the particular user or the skin or hair type associated with that user finds appropriate or desirable. Thus, new products or products not already entered into the identifier can be added to the identifier along with the information concerning the skin or hair type. Also, an embodiment can correlate the ingredients of a product in the identifier and/or in the preferences with ingredients in an “Ingredient” database, so that new products with similar ingredients can also be recommended and/or avoided.

FIG. 2 depicts a chart with sixteen exemplary skin types, Baumann Skin Type, as referred to above. Each of the letters represent one of the four factors or parameters—O (oily) or D (dry); S (sensitive) or R (resistant); P (Pigmented) or N (non-pigmented); W (wrinkled) or T (tight). Based upon a scoring system or upon answers to a questionnaire, an individual can be assigned one of the sixteen skin types depicted in the combinations shown in FIG. 2.

FIG. 3 depicts a chart with sixteen exemplary hair types, Baumann Hair Type, as referred to above. Each of the letters represents one of the four factors or parameters—O (oily) or D (dry) hair; I (injured) or U (uninjured) hair; F (small or fine diameter) or T (large or thick diameter) hair; C (curly) or S (straight) hair. Based upon a scoring system or upon answers to a questionnaire, an individual can be assigned one of the sixteen hair types depicted in the combinations shown in FIG. 3.

FIG. 4 depicts an exemplary operation of the input and output options available in certain embodiments of the invention but the figure is not intended to encompass all possible embodiments. The optional patient/customer information can be collected first and then stored in a separate database or any database. In an option where four factors or parameters are selected, answers to questions or other inputs related to each-of the four parameters or factors determine the score or degree for that factor (for example O or D as mentioned above). The arrows in both directions indicate that questions can be designed to refine the degree or score for that parameter by requiring more than one response or more than one input. The dotted line arrows indicate that information concerning the products or ingredients or combinations of ingredients can be used to help refine the score or degree in any or all or any combination of factors, if desired. Thus, oily skin with an allergy to certain compounds can result in different questions or queries being sent to the user or patient or customer and the different questions may be further changed by the presence of certain products or ingredients in the database that may or may not be recommended. After the four factors have adequate input, a recommendation on a product, an ingredient, a combination of ingredients, or a treatment is made. A recommendation can include a suggestion to NOT use a particular product or ingredient as well as a suggestion to use a particular product or ingredient. This is generally referred to as the output recommendation, which optionally can be stored along with the query session in a separate database or associated with a database or stored with the patient/customer information. In another embodiment, the questionnaire incorporates information that is related to the input or response for a parameter or a factor or is perhaps related to more than one parameter or factor. For example, the questionnaire can include using questions related to a patient's or customer's age, habits such as diet and smoking, skin coloration, dryness, oiliness, sensitivity to compounds or abrasion, sunburn susceptibility, pimple breakout affinity, degree of wrinkle formation, and pore size. Depending on the responses, different questions may be asked or a particular subset of the possible skin types will be eliminated or selected, for example. Thus, as is shown in FIG. 4, the responses to questions or input for any particular factor may also affect the degree or score in another parameter or factor (shown in the interaction through the database at the bottom).

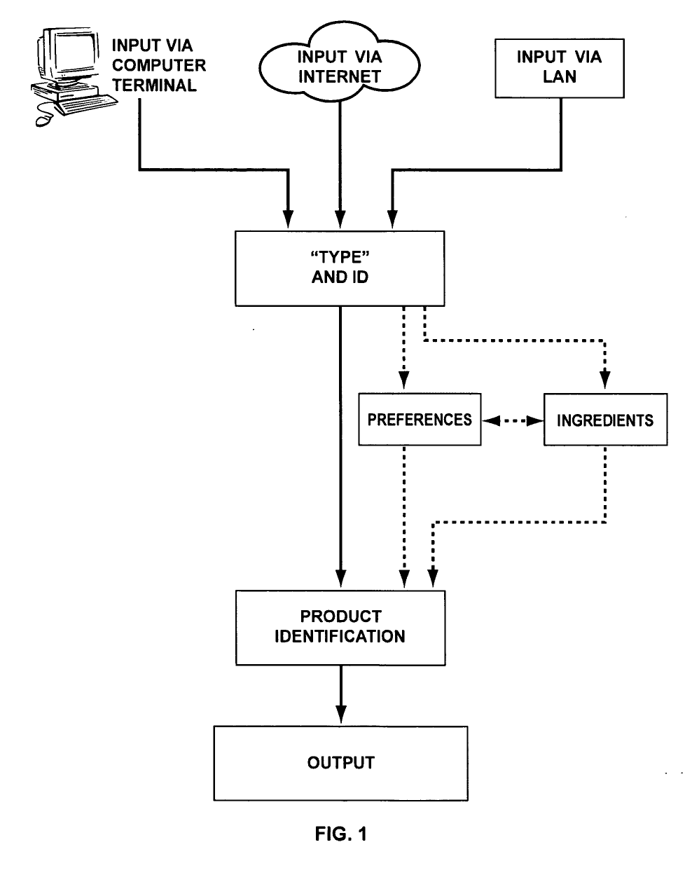

### DESCRIPTION OF THE APPENDICES

The appendices filed on CD contain a Table 1 and Table 2. Table 1 is a listing various ingredients for cosmetic, skin care, and hair care products. In addition, vitamin supplements and other ingredients used as food supplements or therapeutic treatments may be listed as they relate to claimed or associated skin or hair benefits. Each of the ingredients can be associated with a particular indicator (i.e., “recommend,” or “avoid,” or “neutral”) for one or more particular skin types or hair types. Table 2 lists an exemplary questionnaire for determining skin types using a scoring system that gives one of two options for each of four factors.

### DETAILED DESCRIPTION

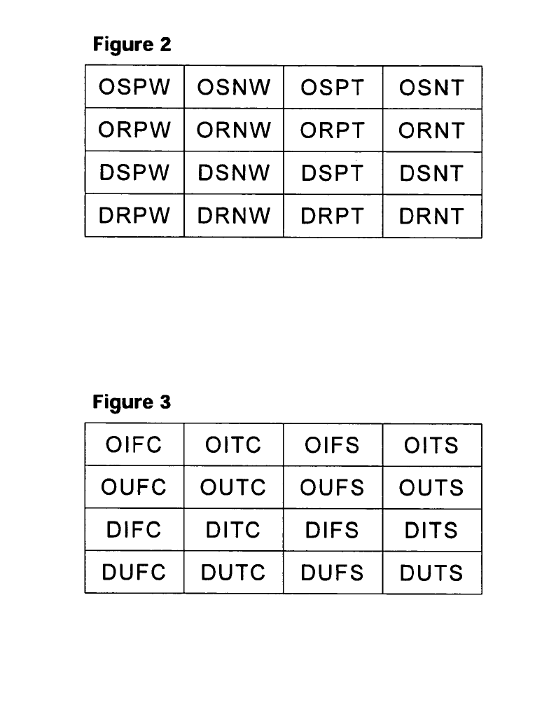

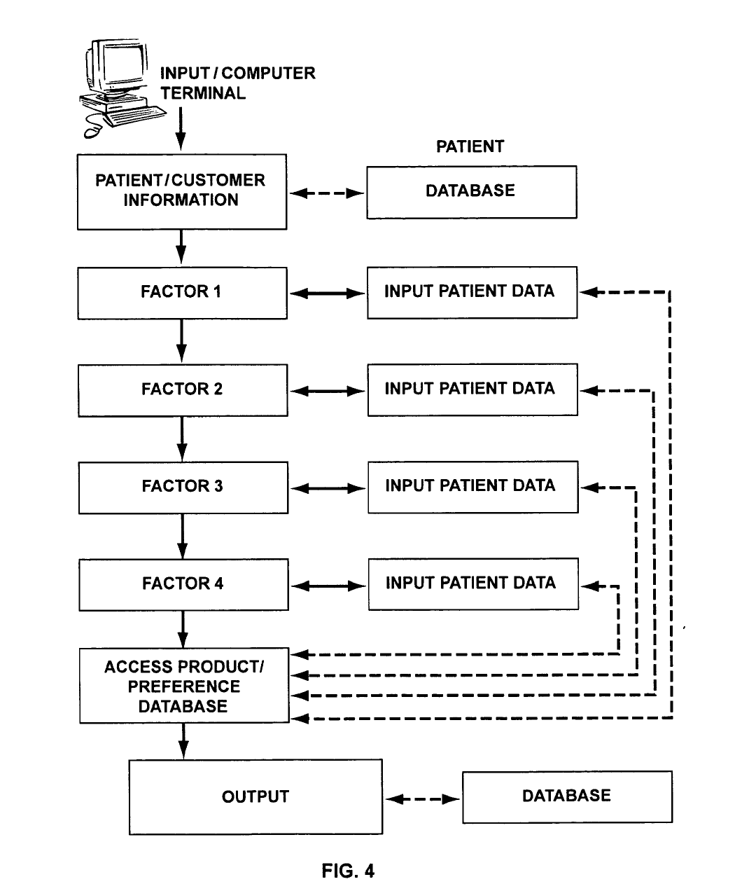

In one aspect, the invention provides a solution to the problem of selecting, recommending, or categorizing skin or hair care products so that professionals, patients or consumers can more accurately or more appropriately select or make recommendations on products that will improve hair or skin. By assigning a skin type or hair type, especially the skin or hair types of FIG. 2 or 3, products can be matched to an individual's skin or hair in ways that prevent undesirable effects and/or improve various conditions in the skin or hair.

In one example, the invention comprises a method of assigning one of sixteen skin types to a potential skin care customer or personal care product user that comprises scoring the skin or a portion of the skin of the customer or user for at least four factors: 1) the degree of oily or dry skin present; 2) the degree of sensitive or resistant skin present; 3) the degree of pigmented or non-pigmented skin present; and 4) the degree of wrinkled or tight skin present. The degree of skin present for each of the factors 1) through 4) can be an either or (i.e., either oily or dry, or either sensitive or resistant), so that each factor has one of two values or scores. However, the score or values can be more than one of two options. Similarly, the invention comprises a method of assigning one of sixteen hair types to a potential hair care customer or personal care product user, comprising scoring the hair of the customer or user for at least four factors: a first factor reflecting the degree of oily (O) or dry (D) hair present; a second factor representing the degree of injured (I) or uninjured (U) hair present; a third factor representing the degree of small diameter or fine (F) hair or large diameter or thick (T) hair present; and a fourth factor representing the degree of curly (C) or straight (S) hair present. The skin types of FIG. 2 and hair types of FIG. 3 reflect four factors where each factor has one of two possible values or scores, and this is the preferred method for scoring skin and hair. A questionnaire can be used to determine which of the two values or scores for each factor is appropriate, and an exemplary questionnaire is presented in the Appendix Table 2. A similar questionnaire can be developed for hair type factors, and the results used to select one of the sixteen hair types in FIG. 3.

In another embodiment, the skin type or hair can be used in a method of recommending a skin care or hair care product to a potential customer. The method can employ an online or computer-based system to input a skin type or hair type, such as one of those shown in FIGS. 2 and 3. The online or computer-based system can then display or communicate one or more products recommended for that skin or hair type, or one or more products having an ingredient that is recommended for the particular skin or hair type. The system can store and routinely update the products that are recommended or should be avoided for each of the hair and skin types. A database or computer-based list of ingredients can also be used, where optionally each product is linked or associated with information on ingredients contained within the product. In one example, one or more products are recommended based upon information about a plurality of skin care products and their one or more ingredients that includes a “recommend” indication associated with a particular skin type.

In another aspect, the invention comprises a questionnaire for assigning one of sixteen skin types or hair types to a person or a section of the person's skin or hair. For the skin type example, the questionnaire comprises questions designed to determine four factors, such as whether oily (O) or dry (D) skin is present, whether sensitive (S) or resistant (R) skin is present, whether pigmented (P) or non-pigmented (N) skin is present, and whether wrinkled (W) or tight (T) skin is present. In the related hair type embodiment, the questionnaire is designed to determine four factors, such as whether oily (O) or dry (D) hair is present, whether injured (I) or uninjured (U) hair is present, whether small diameter or fine (F) hair or large diameter or thick (T) hair is present, and whether curly (C) or straight (S) hair is present. The questionnaire can be presented in paper form, in an online format, or through an electronic device or computer-based system. In one example, the system for identifying a hair or skin care product can comprise an interface for receiving a skin or hair type for an individual, wherein a value or score for at least four factors related to the condition of the skin or hair is used. A computer or network can use a product identifier that compares the skin or hair type to stored information on a set of skin or hair care products or ingredients, wherein the information on a set of products includes for each product or ingredient one or more of: an “avoid” indicator for a particular skin or hair type; a “recommend” indicator for a particular skin or hair type; a “neutral” indicator for a particular skin or hair type. As known in the art, an output interface for displaying or communicating information on the skin or a hair care product identified can be used.

For an online seller of cosmetic or skin or hair care products, the systems and methods of the invention can provide superior suggestions to potential customers and generate improved customer loyalty. Furthermore, it can provide a basis for generating a database, and an updateable database, of preferred products for particular skin or hair types. Thus, not only can the systems and methods used stored information and skin or hair type scores to make a product recommendation, the systems and methods can provide user comments related to the use of certain products for certain skin and hair types. One of skill in the art of online marketing and web-based sales is familiar with the user or customer ID options to associate a particular user with the information entered by that user, thus linking a satisfied user of a product to a particular skin or hair type.

### EXAMPLES

A questionnaire can be used to generate a score or skin or hair type score for an individual. Such a questionnaire can be employed in many forms, including online forms, through network interfaces, brochures, telephone responses, or other forms. In the appendix Table 2, for example, on the oily vs dry portion of the questionnaire, a score of 11 to 44 is possible, with a score between 34-44 representing very oily skin, a score between 27-33 representing slightly oily skin, a score between 17-26 representing slightly dry skin, and a score between 11-16 representing dry skin. Similarly, the other factors can generate numbers in order to assign a score or value for a factor in other skin type factors and hair type factors. Thus, an O or D score can be used, or a score of one of O1 or O2 plus a score of one of D1 or D2 can be used.

For individuals assigned an oily (O) value or score for the oily vs. dry factor in the skin type scoring system, where the factor is only one of two options O or D, the system and method would recommend using a product with benzoyl peroxide, retinol, salicylic acid (or BHA), tea tree oil, and/or zinc for reducing acne. In addition, the ingredients to avoid for the individual with oily skin could be communicated as cinnamon oil, cocoa butter, cocos nucifera (coconut oil), isopropyl isostearate, isopropyl mystirate, peppermint oil, and sodium laurel sulfate. For the individual that in addition has sensitive (S) skin, the system or method could recommend products with: aloe vera, arnica, calendula, chamomille, colloidal oatmeal, cucumber, dexapanthenol, evening primrose oil, feverfew, green tea, licochalone, perilla leaf extract, pycnogenol (pine bark extract, red algae, trifolium pretense (red clover), thyme, epilobium angustifolium (willow herb), and zinc for reducing inflammation. For the individual that in addition has pigmented (P) skin, products with ingredients arbutin, bearberry extract, cucumber, glycyrrhiza glabra (licorice extract), hydroquinone, mulberry extract, and niacinamide can be used to lighten dark spots. For the individual that in addition has wrinkled (W) skin, products with the following ingredients can be used to prevent wrinkles: alpha lipoic acid, basil, caffiene, carrot extract, copper peptide, coenzyme Q10, cucumber, cucumin (tetrahydracucumin or tumeric), ferulic acid, feverfew, ginger, ginseng, grape seed extract, green tea, idebenone, lutien, lycopene, pomegranate, pycnogenol, red clover, rosemary, sulymarin, and yucca. In addition, products to avoid for preventing further pigmentation and wrinkling include those with: avobenzone, benzophenones, methoxycinnamate, para aminobenzoic acid (PABA). Using these “recommend” and “avoid” indicators as an example, any product can be stored along with its ingredients and then compared to a list of ingredients recommended or that should be avoided for a particular skin type. In this way, new products that have not been specifically tested on a particular skin type, or for which no user information is available, can be coded for an indicator as “recommend” “avoid” or “neutral” based solely on the ingredient information. As noted above, one or more of “recommend” or “avoid” or “neutral” indicators can be used in any of the aspects of the invention. The Table 1 list of ingredients is an example of the number of ingredients that could be used in a stored system or network for these and related purposes. Each ingredient can also be electronically linked to or associated with one or more products than contains the ingredient, especially if the amount is likely to or sufficient to cause some effect on skin or hair. Similarly, a large number of product types can be employed in any aspect of the invention, including cleansing or washing products, toner products, facial products, body products, makeup products, sunscreen products, moisturizing products, and the like, and each can be addressed in the methods and systems of the invention.

As another example, hair care products can be associated with the ingredients listed in Table 1 and then stored to recommend or avoid indicators for each of the hair types.

The invention is not limited to any particular embodiment or example given here. Instead, one or skill in the art can use the information and concepts described to devise many other embodiments beyond those given specifically here. In addition, the examples given and embodiments described should not be taken as a limitation to the scope of the claims that follow, and one of skill in the art can use the invention described herein to make and use embodiments that are not specifically described here.

What is claimed is:

1. A method of assigning a diagnostic phenotype to a human user of skin care products wherein
a questionnaire consisting of four sets of questions is administered to the user, wherein each set is a condition-specific pairing of skin parameters, each condition-specific pairing consisting of two phenotypes, said pairings being selected from (i) a phenotype of oily skin (designated by the letter O) or a phenotype of dry skin (designated by the letter D), (ii) a phenotype of sensitive skin (designated by the letter S) or a phenotype of resistant skin (designated by the letter R), (iii) a phenotype of pigmented skin (designated by the letter P) or a phenotype of non-pigmented skin (designated by the letter N), and (iv) a phenotype of tight skin (designated by the letter T) or a phenotype of wrinkled skin (designated by the letter W),
wherein for each set of condition-specific pairings of skin parameters, the user answers a first question for the pairing, the answer to the first question for the pairing is assigned a numerical score and, based on the user's answer to the first question for the pairing, the user is asked at least one follow-up question, where each answer to each of the at least one follow-up question is assigned a numerical score, and the scores for each pairing are totaled and, based on a total score for the skin parameter, the user is assigned a phenotype from each of the four pairings.

2. The method of claim 1 wherein the skin is assigned to one of sixteen, diagnostic skin types, each having a four-letter designation and selected from OSPW, OSNW, OSPT, OSNT, ORPW, ORNW, ORPT, ORNT, DSPW, DSNW, DSPT, DSNT, DRPW, DRNW, DRPT, or DRNT.

3. The method of claim 1 wherein the user is queried to describe her/his ethnicity from (a) African-American/Caribbean/Black (b) Asian/Indian/Mediterranean, (c) Latin-American/Hispanic and (d) Caucasian.

4. The method of claim 3 wherein the set of questions for the sensitive or resistant pairing queries the user about whether the user has or has received a diagnosis of acne, rosacea, contact dermatitis, or eczema.

5. The method of claim 4 wherein the set of questions for the sensitive or resistant pairing queries the user about the frequency with which the user experiences itching, redness, or break outs after applying makeup, sunscreen, or skin care products

6. The method of claim 3 wherein the set of questions for the oily or dry pairing queries the user to describe her/his face two to three hours after washing her/his face, but not applying any moisturizer, sunscreen, toner, powder, or other skin care products, by selecting one of the following four answers, where (a) is very rough, flaky, or ashy, (b) is tight, (c) is no reflection of light and well hydrated and (d) is shiny with reflection of bright light.

7. The method of claim 6 wherein the set of questions for the oily or dry pairing queries the user about one or more of (a) the oiliness of her/his face in the t-zone (forehead and nose) and (b) the frequency she/he has clogged pores (blackheads or whiteheads).

8. The method of claim 3 wherein the set of questions for the tight or wrinkled pairing queries the user about one or both of (i) the frequency with which the user tans from outdoor activities or a tanning bed (ii) the propensity of the user to burn or tan when exposed to sun.

9. The method of claim 8 wherein the set of questions for the tight or wrinkled pairing queries the user about whether she/he has facial wrinkles when smiling, frowning, or lifting her/his eyebrows.

10. A method of assigning a diagnostic skin type to a human user consisting of the steps of administering a questionnaire to the user, said questionnaire assessing four sets of condition-specific questions, wherein the user answers answer denominated “a”, “b”, “c”, “d” or “e”, wherein each questionnaire answer is assigned a numerical score, and the scores for each set of condition-specific questions are totaled to assign the user a diagnostic skin type having four parameters, wherein
(i) a first parameter of the diagnostic skin type assigns a phenotype of oily skin (designated by the letter O) or dry skin (designated by the letter D) and is based on the user's responses to the following eleven questions
(1) two to three hours after washing her/his face, but not applying any moisturizer, sunscreen, toner, powder, or other products, the user is asked to describe the appearance or feel of her/his forehead and cheeks by selecting one of the following four answers, where (a) is very rough, flaky, or ashy, (b) is tight, (c) is no reflection of light and well hydrated and (d) is shiny with reflection of bright light;
(2) the user is asked to select the frequency her/his face appears shiny, from one of four answers, where (a) is never, (b) is sometimes, (c) is frequently (d) is always;
(3) two or three hours after applying a facial makeup foundation, but no powder, the user is asked to describe the appearance of her/his makeup by selecting one of the following five answers, where (a) is flaky or caked in wrinkles, (b) is smooth, (c) is shiny, (d) is streaked and shiny and, if the user does not wear a facial makeup foundation she/he answers (e);
(4) when in a low humidity environment, and the user does not use moisturizers or sun-screen, the user is asked to select one of the following five answers to describe the appearance or feel of her/his facial skin, where (a) is feels very dry or cracks, (b) is feels tight, (c) is feels normal, (d) is looks shiny or the user does not believe she/he needs to use moisturizer, and the user answers (e) if none of the foregoing apply;
(5) when the user looks in a magnifying mirror, she/he is asked how to grade the number of large pores, the size of the end of a pin or greater, that she/he sees by selecting one of the following four answers, where (a) is none, (b) is a few in the t-zone (forehead and nose) only, (c) is many, and (d) is a very large number;
(6) the user is asked to describe her/his facial skin by selecting one of the following four characterizations where (a) is dry, (b) is normal, (c) is combination and (d) is oily;
(7) after cleansing her/his facial skin with a soap that suds, bubbles, and foams vigorously, the user is asked to describe how her/his skin feels after cleansing by selecting one of the following five characterizations, where (a) indicates skin that feels dry or cracks, (b) indicates skin that feels slightly dry but does not crack, (c) indicates skin that feels normal, and (d) indicates skin that feels oily, and where a user does not use soap or other foaming cleansers, because such use makes her/his skin feel dry, the user is instructed to select (a);
(8) if the user does not use a facial moisturizer, she/he is asked to specify the frequency her/his facial skin feels tight by selecting one of the following four responses, where (a) is always (b) is sometimes, (c) is rarely and (d) is never;
(9) the user is asked to specify the frequency she/he has clogged pores (blackheads or whiteheads) by selecting one of the following four responses, where (a) is never (b) is rarely, (c) is sometimes and (d) is always;
(10) the user is asked to specify the frequency her/his face is oily in the t-zone (forehead and nose) by selecting one of the following four responses, where (a) is never (b) is rarely, (c) is sometimes and (d) is always;
(11) two to three hours after applying moisturizer to her/his cheeks, the user is asked to describe the appearance or feel of her/his cheeks by selecting from one of four answers where (a) is very rough, flaky, or ashy, (b) smooth, (c) is slightly shiny, and (d) is shiny and slick, and where the user does not use moisturizer, she/he is instructed to select (d); and wherein the user assigns 1 point for every “a” answer, 2 points for every “b” answer”, 3 points for every “c” answer, 4 points for every “d” answer, and 2.5 points for every “e” answer; and further wherein
the user adds her/his scores, and if the user's total score is between 17-44, the user is diagnosed as having an oily skin phenotype (O), and if the user's score is between 11-16, the user is diagnosed as having a dry skin phenotype (D), and a first parameter of the diagnostic skin type of O or D is assigned; and
(ii) a second parameter of the diagnostic skin type assigns a phenotype of sensitive (designated by the letter S) or resistant (designated by the letter R) and is based on the user's responses to the following eighteen questions
(1) the user is asked to describe the frequency of red bumps on her/his face by selecting one of the following four characterizations, where (a) is never, (b) is rarely, (c) is at least once a month and (d) is at least once a week;
(2) the user is asked to describe the frequency when using skin products that she/he breaks out, or experiences a rash, itch, or sting on her/his face by selecting one of the following five responses, where (a) is never, (b) is rarely, (c) often and (d) always, and the user is instructed to select (e) if she/he does not wear skincare products on her/his face; and
(3) the user is asked to whether she/he has ever been diagnosed with acne or rosacea by selecting one of the following five characterizations, where (a) is no, she/he does not have acne or rosacea (b) the user has been told by a friend or acquaintance that she/he has acne or rosacea, (c) yes, she/he does have acne or rosacea, (d) yes, she/he does have severe acne or rosacea, and (e) if the user is unsure whether she/he has acne or rosacea;
(4) the user is asked to how often she/he gets a rash when wearing jewelry that is not 14-carat gold, by selecting one of the following five responses, where (a) is never, (b) is rarely, (c) is often, (d) is always and (e) is unsure;
(5) the user is asked to how often application of a sunscreen make her/his skin itch, burn, break out, or turn red by selecting one of the following five responses, where (a) is never, (b) is rarely, (c) is often, (d) is always and (e) the user never wears sunscreen;
(6) the user is asked whether she/he has ever been diagnosed with atopic dermatitis, eczema, or contact dermatitis by selecting one of the following five characterizations, where (a) the user does not have atopic dermatitis, eczema, or contact dermatitis (b) the user has been told by a friend that she/he has atopic dermatitis, eczema, or contact dermatitis, (c) the user has atopic dermatitis, eczema, or contact dermatitis, (d) the user has severe atopic dermatitis, eczema, or contact dermatitis and (e) if the user is unsure whether she/he has atopic dermatitis, eczema, or contact dermatitis;
(7) the user is asked to how often she/he gets a rash when wearing jewelry that is not 14-carat gold, by selecting one of the following five responses, where (a) is never, (b) is rarely, (c) is often, (d) is always and (e) is unsure;
(8) the user is asked the frequency that Fragranced bubble bath, massage oil, or body lotions make her/his skin break out, itch, or feel dry, by selecting one of the following five responses, where (a) is never, (b) is rarely, (c) is often, (d) is always and (e) these products are never used, but the user is instructed to select (d) if lack of use or these products is related to skin that breaks out, itches, or feels dry;
(9) the user is queried whether she/he can you use the soap provided in hotels on your body or face without a problem, and responds by selecting one of the following five responses, where (a) is yes, (b) is most of the time, I don't have a problem, (c) is no, my skin itches, turns red, or breaks out, (d) is I would not use hotel soap because of too many problems in the past, and (e) is unsure, because the user travels with her/his own soap when staying in a hotel;
(10) the user is asked whether someone in her/his family has been diagnosed with atopic dermatitis or eczema by selecting one of the following five characterizations, where (a) indicates no member of the user's family has been diagnosed with atopic dermatitis or eczema, (b) indicates one family member has been diagnosed with atopic dermatitis or eczema, (c) indicates several family members have been diagnosed with atopic dermatitis or eczema, (d) indicates many of the user's family members have dermatitis, eczema, asthma, and/or allergies, and (e) is unsure;
(11) the user is asked to describe what occurs if she/he uses scented laundry detergents or static control sheets in the dryer, by selecting one of the following five characterizations, where (a) is, “My skin is fine”, (b) is “My skin feels slightly dry, (c) is “My skin itches, (d) is My skin itches or gets a rash, and (e) is “unsure” because scented laundry detergents or static control sheets in the dryer have not been used;
(12) the user is asked to characterize the frequency her/his face and/or neck gets red after moderate exercise, and/or with stress or a strong emotion, such as anger, by selecting from selecting one of the following four characterizations, where (a) is never, (b) is sometimes, (c) is frequently, and (d) is always;
(13) the user is asked how often she/he gets red and flushed after drinking alcohol by selecting from selecting one of the following five characterizations, where (a) is never, (b) is sometimes, (c) is frequently, and (d) is always, or the user does not drink because she/he becomes flushed and red after drinking alcohol, and (e) the user never drinks alcohol;
(14) the user is asked how often she/he gets red and flushed after eating spicy or hot (temperature) foods or beverages drinking alcohol by selecting from selecting one of the following five characterizations, where (a) is never, (b) is sometimes, (c) is frequently, and (d) is always, and (e) is the user never eats spicy food, but the user is instructed to select (d) if she/he does not eat spicy or hot food because of facial flushing;
(15) the user is asked how many visible red or blue broken blood vessels on her/his face and nose does she/he have, or did she/he have prior to a skincare treatment, by selecting from one of the following four characterizations, where (a) is none, (b) is one to three on entire face including nose, (c) is four to six on entire face including nose, and (d) is more than seven on entire face including nose;
(16) the user is asked to describe how often her/his face looks red in photographs, by selecting from one of the following four characterizations, where (a) is never, or the user has not noticed redness, (b) is sometimes, (c) is frequently, and (d) is always;
(17) the user is queried about the frequency that others ask if she/he is sunburned, even when she/he is not sunburned, by selecting from one of the following five characterizations, where (a) is never, (b) is sometimes, (c) is frequently, and (d) is always, and (e) is “I always am sunburned”;
(18) the user is queried about the frequency she/he experiences redness, itching, or swelling from makeup, sunscreen, or skin care products, by selecting from one of the following five characterizations, where (a) is never, (b) is sometimes, (c) is frequently, and (d) is always, and (e) indicates the user does not use makeup, sunscreen, or skin care products, but the user is instructed to select (d) if she/he does not use these products because of redness, itching, or swelling; and wherein
the user assigns 1 point for every “a” answer, 2 points for every “b” answer”, 3 points for every “c” answer, 4 points for every “d” answer, and 2.5 points for every “e” answer; and further wherein
the user adds her/his scores, and if the user has received a diagnosis of acne, rosacea, contact dermatitis, or eczema from a dermatologist, the user adds 5 points to her/his score, or if the user has received a diagnosis of acne, rosacea, contact dermatitis, or eczema from a physician other than a dermatologist, the user adds 2 points to her/his score, and if the user's total score is between 30-72, the user is diagnosed as having a sensitive skin phenotype (S), and if the user's score is between 17-29, the user is diagnosed as having a resistant skin phenotype (R), and a second parameter of the diagnostic skin type of S or R is assigned; and wherein
(iii) a third parameter assigns a phenotype of pigmented (designated by the letter P) or non-pigmented (designated by the letter N) and is based on the user's responses to the following thirteen questions
(1) after she/he has a pimple or ingrown hair, the user is queried about the frequency it is followed by a dark, brownish/black spot, and is asked to select from one of the following five characterizations, (a) never, (b) sometimes, (c) frequently, (d) always and (e) the user never gets pimples or ingrown hairs;
(2) the user is asked to describe how long a brown, not pink, mark remains after the user cuts herself/himself by selecting one of the following four characterizations, (a) the user does not develop a brown mark, (b) a week, (c) a few weeks, and (d) months
(3) the user is queried about the number of dark spots the appeared on her face when she was pregnant, using birth control pills, or taking hormone replacement therapy and is asked to select one of the following five answers, where (a) is none, (b) is one, (c) is a few, (d) is a lot, and (e) indicates that the question is not applicable because the user is male or has never been pregnant, or taken birth control pills, or taken hormone replacement therapy;
(4) the user is queried about whether she/he has any dark spots or patches on the upper lip or cheeks, or whether she/he had any dark spots or patches on the upper lip or cheeks that were removed where, and the user is asked to select one of the following four answers (a) is none, (b) indicates the user is uncertain, (c) indicates the dark spots or patches are, or were, slightly noticeable, (d) indicates the dark spots or patches are, or were, very noticeable;
(5) in response to a question whether dark spots on her/his face become more pronounced when exposed to the sun, the user is asked to select one of the following five characterizations, where (a) the user has no dark spots, (b) user is unsure, (c) the dark spots become “slightly worse”, (d) the dark spots become “a lot worse” and (e) the user wears sunscreen on her/his face every day, but if the user uses sunscreen daily because of concern about developing dark patches or freckles, the user is instructed to answer (d);
(6) the user is queried about whether she/he has been diagnosed with melasma, tan or brown patches, on her/his face and is asked to select one of the following four responses, where (a) is no, (b) is once, but the melasma, tan or brown patches went away, (c) is yes, (d) is yes, a servere case and (e) the user is unsure;
(7) the user is queried about the number of small brown spots (freckles or sun spots) on her/his face, chest, back, or arms and is asked to select one of the following five response, where (a) is none, (b) is one to five, (c) is six to fifteen and (d) is sixteen or more;
(8) when exposed to sun for the first time in several months, the user is asked to characterize her/his skin's response from one of the following four responses (a) burns only, (b) burns then gets darker, (c) gets darker, and (d) the user's skin is already dark so it is hard to see if it gets darker.
(9) when the user is exposed to many consecutive days of sun exposure, she/he is asked to characterize her/his skin's response from one of the following five responses (a) the user's skin sunburns and blisters, but her/his skin does not change color, (b) the user's skin becomes slightly darker, (c) the user's skin becomes much darker, (d) the user's skin is already dark so it is hard to see if it gets darker, and (e) unsure;
(10) the user is queried about the number of freckles, defined as small 1-2 mm, pinpoint-sized flat spots, that she/he develops when exposed to sun, and the user is asked to characterize her/his skin's response from one of the following five responses (a) the user never develops freckles, (b) the user develops a few new small freckles each year, (c) the user develops new freckles often, (d) the user's skin is already dark so it is hard to see if she/he has freckles, and (e) the user does not go in the sun;
(11) the user is asked to describe whether either of her/his parents have freckles, and, if so, the number of freckles by selecting one of the following five characterizations (a) neither parent had freckles (b) one or both parent(s) had a few freckles on the face, (c) one or both parent(s) had a many freckles on the face, (d) one or both parent(s) had a many freckles on the face, chest, neck, and shoulders and (e) unsure;
(12) the user is asked to describe her/his natural hair color from the group consisting of (a) blonde, (b) brown, (c) black and (d) red;
(13) the user is asked to describe whether she/he or any of her/his immediate family members have a history of melanoma by selecting one of the following five response (a) no, (b) one immediate family member, (c) more than one immediate family member, (d) the user has melanoma, and (e) unsure; and further wherein
the user assigns 1 point for every “a” answer, 2 points for every “b” answer”, 3 points for every “c” answer, 4 points for every “d” answer, and 2.5 points for every “e” answer; and further wherein
the user adds her/his scores, and if the user has dark spots on her/his skin in areas of sun exposure, the user add five points to her/his score, and if the user's total score is between 29-52, the user is diagnosed as having a pigmented phenotype (P), and if the user's score is between 13-28, the user is diagnosed as having a non-pigmented phenotype (N), and a third parameter of the diagnostic skin type of P or N is assigned; and
(iv) a fourth parameter of the diagnostic skin type assigns a phenotype of wrinkled skin (designated by the letter W) or tight skin (designated by the letter T) and is based on the user's responses to the following twenty questions,
(1) when asked whether she/has facial wrinkles, the user selects one of the following five responses, (a) no wrinkles, not even with facial muscle movements such as smiling, frowning, or lifting my eyebrows, (b) wrinkles only with facial movements, such as smiling, frowning, or lifting my eyebrows, (c) wrinkles with facial muscle movement and a few wrinkles when the user is not smiling, frowning, or lifting brows, (d) wrinkles are present even if the user is not smiling, frowning, or lifting brows;
(2) when asked about the appearance of her/his mother's facial skin, the user selects one of the following five responses (a) five to ten years younger than her age, (b) her age, (c) five years older than her age, (d) more than five years older than her age, or (e) not applicable, because the user was adopted or cannot remember;
(3) when asked about the appearance of her/his father's facial skin, the user selects one of the following five responses (a) five to ten years younger than his age, (b) his age, (c) five years older than his age, (d) more than five years older than his age, or (e) not applicable, because the user was adopted or cannot remember;
(4) when asked about the appearance of her/his maternal grandmother's facial skin, the user selects one of the following five responses, the maternal grandmother looked (a) five to ten years younger than her age, (b) her age, (c) five years older than her age, (d) more than five years older than her age, or (e) not applicable, because the user was adopted or cannot remember;
(5) when asked about the appearance of her/his maternal grandfather's facial skin, the user selects one of the following five responses, the maternal grandfather looked (a) five to ten years younger than his age, (b) his age, (c) five years older than his age, (d) more than five years older than his age, or (e) not applicable, because the user was adopted or cannot remember;
(6) when asked about the appearance of her/his paternal grandmothers facial skin, the user selects one of the following five responses, the paternal grandmother looked (a) five to ten years younger than her age, (b) her age, (c) five years older than her age, (d) more than five years older than her age, or (e) not applicable, because the user was adopted or cannot remember;
(7) when asked about the appearance of her/his paternal grandfather's facial skin, the user selects one of the following five responses, the paternal grandfather looked (a) five to ten years younger than his age, (b) his age, (c) five years older than his age, (d) more than five years older than his age, or (e) not applicable, because the user was adopted or cannot remember;
(8) when asked about the number of years in which the user's skin was tanned for more than two times per week, the user selects from one of the four following responses (a) never, (b) one to five years, (c) five to ten years, and (d) more than ten years; and
(9) when asked whether she/he has tanned two weeks per year or less, the user selects from one of the four following responses (a) never, (b) one to five years, (c) five to ten years, and (d) more than ten years;
(10) when asked how much lifetime sun exposure the user experienced, the user selects one of the four following responses (a) little, (b) some, (c) moderate and (d) a lot;
(11) when asked how the user thinks she/he looks, the user responds by selecting (a) one to five years younger than her/his age, (b) her/his age, (c) five to ten years older than her/his age, and (d) more than ten years older than her/his age;
(12) when asked to characterize the frequency during the last five years the user has allowed her/his skin to tan either intentionally or unintentionally through outdoor sports or other activities, the user responds by selecting (a) never, (b) once a month, (c) once a week or (d) daily;
(13) when asked to characterize the frequency the user has been on a tanning bed, the user responds by selecting (a) never, (b) one to five times, (c) five to ten times or (d) many times;
(14) when asked how many cigarettes she/he has smoked, or been exposed to, the user responds by selecting (a) none, b) a few packs, (c) several to many packs, (d) she/he smoke everyday, (e) the user has never smoked but has lived, been raised by, or worked with people who regularly smoke in her/his presence;
(15) the user is asked to characterize the air quality where she/he resides from (a) the air is fresh and clean all of the year, (b) the air is fresh and clean for part, but not all of the year, (c) the air is slightly polluted, (d) the air is very polluted;
(16) the user is asked to describe the frequency of her/his use of facial creams containing a retinoid by selecting (a) many years, (b) occasionally, (c) once for acne when I was younger and (d) never;
(17) the user is asked to describe frequency with which she/he eats fruits and vegetables by selecting (a) at every meal, (b) once a day, (c) occasionally, and (d) never;
(18) the user is asked to describe over her/his lifetime, the percentage of her/his daily diet that has consisted of fruits and vegetables from (a) 75-100 percent, (b) 25-75 percent, (c) 10-25 percent, and (d) 0-10 percent;
(19) the user is asked to describe her/his natural skin color from (a) dark, (b) medium tone, (c) light, (d) very light;
(20) the user is asked to describe her/his ethnicity from (a) African-American/Caribbean/Black, (b) Asian/Indian/Mediterranean, (c) Latin-American/Hispanic and (d) Caucasian; wherein
the user assigns 1 point for every “a” answer, 2 points for every “b” answer”, 3 points for every “c” answer, 4 points for every “d” answer, and 2.5 points for every “e” answer; and further wherein
the user adds her/his scores, and if the user is sixty-five years of age or older, the user add five points to her/his score, and if the user's total score is between 20-40, the user is diagnosed as having an pigmented phenotype (T), and if the user's score is between 41-85, the user is diagnosed as having a non-pigmented phenotype (W), and a fourth parameter of the diagnostic skin type of W or T is assigned.

11. A method of assigning a diagnostic skin type skin selected from sixteen, diagnostic skin types, each having a four-letter designation and selected from OSPW, OSNW, OSPT, OSNT, ORPW, ORNW, ORPT, ORNT, DSPW, DSNW, DSPT, DSNT, DRPW, DRNW, DRPT, or DRNT to a human user consisting of the steps of administering a questionnaire to the user, said questionnaire assessing four sets of condition-specific questions, in which the answers are input into, and a score is output by, via an electronic device, a terminal or a computer, or through an online network.

12. The method of claim 11 wherein the questionnaire is self-administered by the user.

13. The method of claim 11 wherein the questionnaire is administered by a physician, a nurse, an aesthetician, or a skin care professional.
